(19) United States
(12) Patent Application Publication
(10) Pub. No.: US 2024/0255407 A1
(43) Pub. Date: Aug. 1, 2024

(54) METHOD AND SYSTEM FOR DETERMINING MAGNETIC BEAD CONCENTRATION IN SUSPENSION

(71) Applicant: SEEGENE, INC., Seoul (KR)

(72) Inventors: Hyun Jin LIM, Seoul (KR); Deog Pill CHO, Seoul (KR); Yoo Jun HAN, Gyeonggi-do (KR); Won Seok JEONG, Seoul (KR); Huy Youel CHOI, Gyeonggi-do (KR)

(21) Appl. No.: 18/289,693

(22) PCT Filed: May 3, 2022
(86) PCT No.: PCT/KR2022/006362

§ 371 Date: Nov. 6, 2023

(30) Foreign Application Priority Data
May 7, 2021 (KR) 10-2021-0059334

(51) Int. Cl. G01N 15/075; G01N 15/00

(52) U.S. Cl. CPC G01N 15/075; G01N 2015/0053

(57) ABSTRACT

Provided is a method and system for determining a magnetic bead concentration in a suspension according to the disclosure, including irradiating a plurality of different wavelengths of light to a suspension in which magnetic beads are suspended, measuring the intensities of a plurality of reflected light beams at the different wavelengths reflected from magnetic beads, and determining the concentration of the magnetic beads from the measured intensities of the reflected light beams at the different wavelengths.

### TECHNICAL FIELD

The disclosure provides a method and system for determining a magnetic bead concentration in a suspension that irradiates a plurality of light beams having different wavelengths to the suspension through an illuminator, measures the intensities of the light beams reflected at the different wavelengths by the suspension, and determines the concentration depending on the degree of particles suspended in the suspension.

### BACKGROUND ART

In recent years, as image processing technology is advanced and hardware platforms for image processing are developed, vision inspection is essential for various factory automation facilities. Vision inspection is performed by an illuminator for irradiating light, a camera for capturing the target object, and an inspector for inspecting the image obtained by the camera. In a state in which the target object which has undergone a manufacturing process is irradiated with light, capturing is carried out to obtain an image. The obtained image is analyzed to inspect defects in the processed product.

A vision inspection system is used to irradiate light and captures a solution by way of an illuminator and a camera to measure the concentration based on the color of the solution.

Conventional vision inspection systems primarily measure the concentration by detecting the intensity of light proportional to the concentration according to the color change for the solution whose color is changed depending on the degree at which a solvent or pH indicator is dissolved in a liquid.

However, the solution may contain various components, such as cells or other insoluble solid particles, as well as the solvent or liquid soluble or mixed well, and it is needed to measure the concentration that may differ depending on the content thereof.

Therefore, a need exists for an inspection scheme capable of precisely measuring the concentration according to the characteristics (color, reflectivity, etc.) of the target object.

### DISCLOSURE OF INVENTION

### Technical Problem

The inventors have tried to develop a technique capable of determining the concentration of a suspension by irradiating a solid particle-suspended suspension and measuring the intensity of the light reflected from the solid particles in a vision inspection system.

As a result, the inventors have figured out that it is possible to irradiate a plurality of light beams having different wavelengths, determine a tiny change in concentration by analysis of the reflected light beams at the different wavelengths reflected in response thereto, and image it based on the degree of color.

In the foregoing background, the disclosure provides a method and system for irradiating a plurality of light beams having different wavelengths to a solid particle-suspended suspension, measuring the intensity of the per-wavelength light beams reflected in response thereto, and determining the concentration of the suspension based on the measured per-wavelength intensities.

### Solution to Problem

The disclosure relates to a method for determining the concentration of magnetic beads in a suspension.

In an example, the method comprises irradiating a plurality of light beams having different wavelengths to a plurality of wells containing a suspension in which magnetic beads are suspended, sensing reflected light beams at the different wavelengths from the plurality of wells using a detector including a light sensor, and determining concentrations of the magnetic beads in the plurality of wells by analyzing light data of the sensed reflected light beams at the different wavelengths.

In an example, sensing the reflected light beams at the different wavelengths includes measuring intensities of the reflected light beams at the different wavelengths.

In an example, the plurality of light beams are selected from among light beams reflectable by the magnetic beads.

In an example, the plurality of light beams are two or more light beams selected from among ultraviolet (UV) light, blue light, green light, orange light, red light, far-red light, infrared light, and white light. The plurality of light beams are sequentially irradiated.

In an example, determining the concentrations of the magnetic beads includes calculating a certain concentration value or a concentration range of the magnetic beads in the wells or identifying whether the concentrations of the magnetic beads in the wells fall within a predetermined range, using the light data of the reflected light beams at the different wavelengths.

In an example, analyzing the light data of the sensed reflected light beams at the different wavelengths includes determining the concentration of the magnetic beads in the plurality of wells, using a standard curve of per-wavelength reflected light intensities for magnetic beads for a plurality of known concentrations.

In an example, the method further comprises identifying whether respective intensities of the reflected light beams at the different wavelengths fall within reference intensity ranges respectively provided for the wavelengths and determining that the magnetic beads in the wells fall within a desired concentration range if all of the reflected light beams at the different wavelengths fall within the reference intensity ranges, if a predetermined number of, or more, reflected light beams at the different wavelengths fall within the reference intensity ranges, or if reflected light beams having two or more pre-selected wavelengths fall within the intensity ranges.

In an example, the detector is a colorimeter.

In an example, the detector senses the reflected light beams per wavelength by a monochrome camera and provides an image for the sensed reflected light beam per wavelength.

In an example, irradiating the plurality of light beams includes irradiating the light beams having the different wavelengths according to on-off combinations by a control signal from a controller. Sensing the reflected light beams at the different wavelengths includes driving the detector in synchronization with the irradiation by the controller.

In an example, the magnetic beads include iron oxide.

In an example, the magnetic beads are red-brown iron oxide particles.

In an example, the magnetic beads have a size of 0.1 μm to 6 μm.

The disclosure also relates to a system for determining a concentration in a suspension.

In an example, the system comprises an illuminator irradiating a plurality of light beams having different wavelengths to the suspension, a detector sensing reflected light beams at the different wavelengths from the suspension to which the plurality of light beams are irradiated by the illuminator and a controller controlling the illuminator to irradiate the light beams having the different wavelengths according to on-off combinations, driving the detector according to a control signal synchronized with the control signal, measuring intensities of the reflected light beams at the different wavelengths by analyzing light data of the reflected light beams at the different wavelengths sensed by the detector, and determining a concentration of the suspension.

In an example, the controller calculates a certain concentration value or a concentration range of the magnetic beads in the wells or identifies whether the concentrations of the magnetic beads in the wells fall within a predetermined range, using the light data of the reflected light beams at the different wavelengths.

In an example, the controller uses a standard curve of per-wavelength reflected light intensities for magnetic beads for a plurality of known concentrations.

In an example, the controller identifies whether respective intensities of the reflected light beams at the different wavelengths fall within intensity ranges respectively provided for the wavelengths and determines that the magnetic beads in the wells fall within a desired concentration range if all of the reflected light beams at the different wavelengths fall within the intensity ranges, if a predetermined number of, or more, reflected light beams at the different wavelengths fall within the intensity ranges, or if reflected light beams having two or more pre-selected wavelengths fall within the intensity ranges.

In an example, the suspension is a solution in which magnetic beads are suspended.

### Advantageous Effects of Invention

According to an embodiment of the disclosure, a method for determining the concentration of magnetic beads in a suspension may recognize a tiny color difference depending on the concentration level from a high to low concentration according to the content of the magnetic beads suspended in the suspension using a plurality of different wavelengths of light. Thus, it is possible to provide a desired concentration of magnetic bead-suspended suspension.

### BRIEF DESCRIPTION OF DRAWINGS

FIG. 1 is a flowchart illustrating a method for determining a magnetic bead concentration in a suspension according to the disclosure;

FIG. 2 is a view illustrating an example of a result of measuring per-wavelength intensities of reflected light and an image sensed by irradiating light to a plurality of wells by applying a method for determining a magnetic bead concentration in a suspension according to the disclosure; and

FIGS. 3 and 4 are views illustrating a configuration of a system for implementing a method for determining a magnetic bead concentration in a suspension according to the disclosure.

### MODE FOR THE INVENTION

The disclosure is now described in further detail in connection with embodiments thereof. The embodiments are provided merely to specifically describe the disclosure, and it is obvious to one of ordinary skill in the art that the scope of the disclosure is not limited to the embodiments.

Such denotations as “first,” “second,” “A,” “B,” “(a),” and “(b),” may be used in describing the components of the disclosure. These denotations are provided merely to distinguish a component from another, and the essence of the components is not limited by the denotations in light of order or sequence. When a component is described as “connected,” “coupled,” or “linked” to another component, the component may be directly connected or linked to the other component, but it should also be appreciated that other components may be “connected,” “coupled,” or “linked” between the components.

Determining a magnetic bead concentration in a suspension according to the disclosure may include irradiating a plurality of different wavelengths of light to a suspension in which magnetic beads are suspended, measuring the intensities of a plurality of reflected light beams at the different wavelengths reflected from magnetic beads, and determining the concentration of the magnetic beads from the measured intensities of the reflected light beams at the different wavelengths.

The suspension according to the disclosure may include dispersion.

The magnetic bead according to the disclosure may include a metal oxide or may include a material other than the metal oxide. For example, since the magnetic beads preferably include a ferromagnetic or superparamagnetic material, the magnetic beads may include a metal, e.g., a transition metal. The magnetic beads may include carbon nanofibers.

According to an embodiment, the metal oxide may include an oxide of at least one selected from the group consisting of iron, nickel, niobium, manganese, zinc, chromium, and cobalt.

According to an embodiment, the size of the magnetic beads is not particularly limited and may be selected and applied according to the use. Preferably, the magnetic beads of the disclosure may have a size of 0.1, 0.2, 0.3, 0.4, 0.5, 0.6, 0.7, 0.8, 0.9 μm or less or may have a size of 1, 2, 3, 4, 5, 6, 7 μm or less or more, but is not limited thereto.

According to an embodiment, the magnetic beads may include a combination of two or more types of magnetic beads which are different from each other in material, particle diameter, particle size distribution, shape, or shape.

According to an embodiment, the magnetic beads may include a silica coating layer on the surface. The magnetic beads preferably have a modified surface to increase the binding force with the detection material and, by being surface-modified with the silica coating layer, allow various organic molecules to be easily attached thereto while remaining in a stable state.

As used herein, ‘magnetic bead’ means a particle or a bead that reacts with a magnetic field. In general, magnetic bead refers to a material that does not have a magnetic field but forms a magnetic dipole that is exposed to a magnetic field. For example, magnetic bead refers to a material that may be magnetized under a magnetic field but does not have magnetism on its own in the absence of a magnetic field.

Magnetism used herein may include, but is not limited to, both paramagnetic and superparamagnetic materials.

The magnetic beads according to the disclosure may preferably be beads having a property of binding to a nucleic acid and may be, e.g., a form having a functional group binding to a nucleic acid, such as a —COOH group, but is not limited thereto.

Since the magnetic beads have a property of binding to nucleic acids, the magnetic beads are used in the process of sequentially mixing a sample and various reagents and removing residues other than nucleic acids in sample processing for extracting nucleic acids from a sample.

Sample processing refers to a series of processes to primarily separate an analyte from the sample to thereby obtain a material in the state capable of detection reaction. The term ‘sample processing’ may be used as further meaning the process of detecting a target analyte from the substance in the detection reaction-capable state. The analyte may be, for example, a nucleic acid.

According to an embodiment of the disclosure, the sample processing may include the process of extracting a nucleic acid. To extract nucleic acids, such processes need to be first performed as separating nucleic acids from cells or biological materials, washing the separated nucleic acids to purify them into high-purity nucleic acids.

Specifically, cell lysis is performed by rotating the magnetic beads in a reagent containing the magnetic beads, thereby separating the nucleic acids. Before obtaining the magnetic beads from the magnetic beads-containing reagent, the magnetic beads are washed.

The washing of the magnetic beads may be performed using a 50 to 90% (v/v) ethanol solution, specifically, an 80 to 90% (v/v) ethanol solution.

This washing step may be performed by placing the magnetic beads under a magnetic stand, collecting the magnetic beads, then removing the supernatant, adding a washing buffer, and washing them. Such washing step may be performed one or more times.

After the washing step is optionally performed, the magnetic beads may be separated, and nucleic acids may be separated from the magnetic beads by adding an elution buffer to the separated magnetic beads.

Various reagents necessary for sample processing, e.g., the lysis buffer, the binding buffer, the wash buffer, and the elution buffer, and the magnetic beads put and suspended in the various reagents may be configured in the form of a cartridge.

The cartridge includes a plate including a plurality of wells receiving the solution. In the case of a plate having a plurality of wells, various reagents may be received per well and the magnetic beads are suspended in at least one reagent of the reagents received in the wells.

An appropriate amount of magnetic beads need to be suspended in, e.g., the wash buffer for washing the magnetic beads and the nucleic acids attached to the magnetic beads, among the magnetic bead-suspended suspensions, so as to efficiently perform washing.

In this case, suspension of too many magnetic beads in the wash buffer may lead to an increase in costs and disturbance to separation of the nucleic acids from the magnetic beads in the next sample processing step. In contrast, suspension of too few magnetic beads may deteriorate the efficiency of nucleic acid extraction. Accordingly, an appropriate amount of magnetic beads needs to be suspended, and various methods are attempted to inspect the content of magnetic beads in the magnetic bead-suspended suspension.

Conventionally, an inspector checks the size of magnetic beads with the naked eye to measure the content of the magnetic beads in the magnetic bead-suspended suspension. To that end, all of the magnetic beads should be settled to recognize the size of the magnetic beads. Thus, a wait time is consumed until the suspended magnetic beads are settled. The size check using the naked eye is inappropriate in content inspection due to a significant difference in result from inspector to inspector.

To address such issue, a method for determining a magnetic bead concentration in a suspension according to the disclosure irradiates a plurality of wavelengths of light to the magnetic bead-suspended suspension, senses light reflected from the magnetic beads, analyzes the color of the suspension, and determines the concentration of the magnetic beads.

Although sample processing for extracting nucleic acids using magnetic beads according to the disclosure are exemplified in connection with the method for determining a magnetic bead concentration in a suspension according to an embodiment of the disclosure, the disclosure is not limited thereto, and various changes may be made thereto to be suited for the purpose of inspecting the content of particles in a suspension in which the particles, e.g., magnetic beads, are suspended.

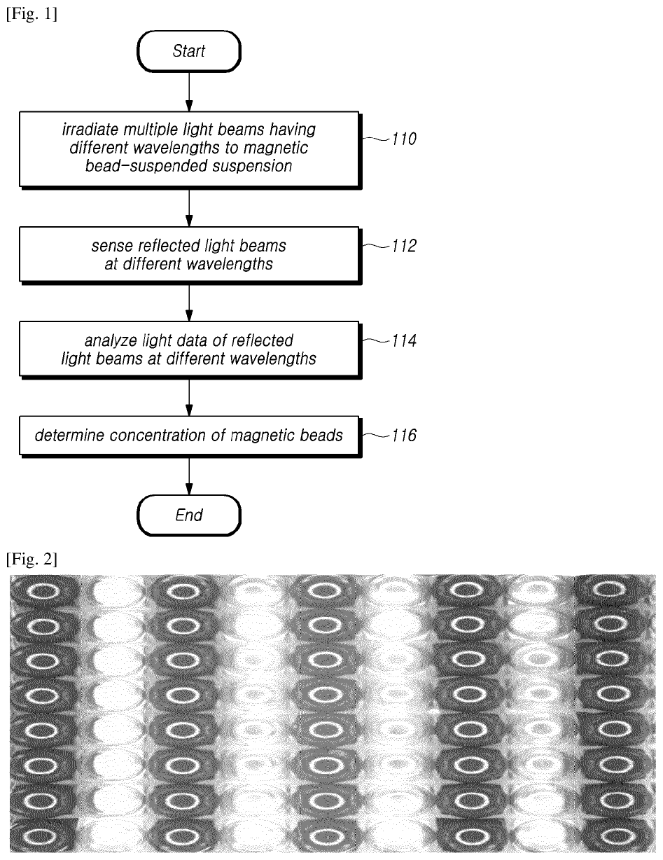

FIG. 1 is a flowchart illustrating a method for determining a magnetic bead concentration in a suspension according to the disclosure.

Referring to FIG. 1, in step 100, a plurality of light beams having different wavelengths are irradiated to a plurality of wells containing a suspension in which objects, preferably, magnetic beads, are suspended.

Radiating the plurality of light beams in step 110 are performed according to on-off combinations for the light beams having the different wavelengths, by a control signal from a controller.

In step 112, reflected light beams at the different wavelengths are sensed from the plurality of wells using a detector including a light sensor. Sensing the reflected light beams at the different wavelengths in step 112 includes driving the detector according to a control signal synchronized with the control signal.

According to an embodiment, the plurality of wells preferably include a plurality of wells formed in a plate. The plate includes a plurality of wells arrayed in a designated matrix.

The number of wells in the plate is not particularly limited and may be, e.g., 2, 3, 4, 5, 6, 7, 8, 9 or more, or may be 1000, 900, 800, 700, 600, 500, 400, 384, 300, 200, 100, 96, 48, 32, 24, 16 or less.

According to an embodiment of the disclosure, all of the wells in the plate have the same size and have a constant inter-well interval. The well is preferably rectangular and should be free from disturbance to image analysis and light irradiation, absorption, transmission, and reflection in the wells containing various reagents due to contamination.

Further, although it has been described that a plurality of wells are formed in the plate according to the disclosure, the disclosure is not limited thereto. Instead of the plate, any member having a different shape from the plate and having at least two wells, tubes, or containers capable of receiving a solution may be used as long as it is able to receive the magnetic bead-suspended suspension and does not disturb image analysis and light irradiation, absorption, transmission, and reflection.

According to an embodiment of the disclosure, in step 110, the plurality of wells are simultaneously irradiated by an illuminator.

Typically, one perceives the color of an object as the light reflected from the object is transferred to the brain. In the disclosure, the color of the magnetic bead-suspended suspension is the color of the light transmitted through the suspension and reflected. Since light reflection increases depending on the amount of magnetic beads suspended in the suspension, the disclosure irradiates wavelengths of light to the magnetic bead-suspended suspension, measures the intensities of the per-wavelength light beams reflected by the magnetic beads to determine the degree of suspension of the magnetic beads, and based thereupon, determines the concentration of the magnetic beads.

Accordingly, in step 110, the plurality of light beams are selected from among light beams reflectable by the magnetic beads.

According to an embodiment, the illuminator according to the disclosure may irradiate different wavelengths of light to the plurality of wells.

Preferably, the plurality of light beams according to an embodiment of the disclosure may be two or more light beams selected from among ultraviolet (UV) light, blue light, green light, orange light, red light, far-red light, infrared light, and white light, and wherein the plurality of light beams are sequentially irradiated.

According to an embodiment of the disclosure, the illuminator may irradiate a single wavelength of white light to the plurality of wells.

In the disclosure, the wavelength includes at least one specific wavelength or a wavelength range. For example, the wavelength may be 400 nm or may be a wavelength range from 390 nm to 410 nm.

According to an embodiment, the different wavelengths may be selected from among wavelengths designated by the user or preset by the controller. The type and number of wavelengths may vary depending on the type of the detector that senses a plurality of per-wavelength light beams.

In the disclosure, the detector may be a colorimeter, and the colorimeter may include a monochrome camera and a red-green-blue (RGB) color camera.

For example, when the detector is an RGB color camera, the illuminator may irradiate a single wavelength of white light and may sense the irradiated white light through filters of red light, green light, and blue light.

When the detector is a monochrome camera, the illuminator may sense each of light beams including UV light, blue light, green light, orange light, red light, far-red light, infrared light, and white light.

In general, when the detector addresses with a color code, it may create different colors by changing the ratio of colors, such as red, green, and blue. After white light is transmitted where different colors are combined, if the intensities of the transmitted light beams in different wavelength ranges are combined, an image in a color perceived by the human eyes may be obtained.

As the number of different wavelength ranges of light to be combined or mixed increases, the color of the image becomes closer to a set of primary colors. For example, as the number of wavelength ranges increases from 3 to 5, from 5 to 7 or 8, an image closer to a set of primary colors may be obtained.

To that end, the monochrome camera senses a single color, e.g., a gray, without filtering per wavelength for the plurality of per-wavelength light beams.

This is why use of a monochrome camera may represent the degree of color, i.e., chromaticity, for various per-wavelength light beams as compared with representing a color using only RGB through an RGB filter in a color camera.

For example, shade information may be obtained from a sensed gray image for each wavelength, and a color may be allocated to the gray image using the obtained shade information so that the degree of shade may be represented for each wavelength, and it is apparent to one of ordinary skill in the art that such method uses well-known pseudo colors.

In the disclosure, if the illuminator irradiates a single wavelength of light, e.g., white light, a color camera may be used and, if a plurality of light beams having multiple wavelengths are irradiated, a color camera or a monochrome camera may selectively be used depending on the characteristics of the wavelengths.

The detector of the disclosure is preferably a monochrome camera, and the illuminator sequentially or simultaneously irradiates multiple wavelengths of light beams including ultraviolet (UV), blue, green, orange, red, far-red, infrared, and white light.

In this case, each wavelength from 380 nm to 890 nm may be irradiated at a preset time interval or simultaneously so that the irradiated wavelength may include all of wavelength ranges including UV (380-450 nm), blue (420-500 nm), green (480-580 nm), orange (530-730 nm), red (630-680 nm), far-red (680-760 nm), infrared (780-890 nm) and white (430-760 nm).

In step 112, in response to irradiation of such wavelengths of light to the plurality of wells, reflected light beams at the different wavelengths are emitted from the plurality of wells and are sensed.

In step 112 for sensing, the intensities of the reflected light beams at the different wavelengths are measured.

Since the illuminator irradiates the above-described wavelength ranges of light to the magnetic bead-suspended suspension, and the detector may receive the reflected light beams, it is possible to measure the intensities of the reflected light beams at the different wavelengths reflectable by the magnetic beads.

In step 114, light data for the sensed reflected light beams at the different wavelengths is analyzed and, in step 116, the concentration of the magnetic beads in the plurality of wells is determined.

Steps 114 and 116 are described below in detail with reference to FIG. 2.

FIG. 2 illustrates a screen displaying an image obtained by irradiating light to a plurality of wells and sensing the reflected light according to the disclosure.

The screen shown in FIG. 2 is an image captured for the bottom of a plate with a plurality of wells.

Although an image captured for the bottom of a plate is exemplified for convenience of description, this is only intended for easy comparison between suspensions containing different amounts of magnetic beads in a plate with a plurality of wells, and it should be noted that the area captured by the detector of the disclosure is not limited thereto.

The image shown in FIG. 2 is a merged image of the images of a plurality of reflected light beams at the different wavelengths captured through the detector according to the disclosure.

The detector according to the disclosure may provide images of reflected light beams at the different wavelengths, per wavelength, or provide a merged image of the plurality of images, using a multi-spectrum image sensor having a pixel array for detecting images having light components in different wavelength ranges.

The detector of the disclosure may capture images of reflected light beams at the different wavelengths using a monochrome camera according to an embodiment, merge the captured images of reflected light beams at the different wavelengths into a single image and display the single image as shown on the left side of FIG. 2.

To that end, the detector according to the disclosure should be able to obtain a combination of the intensities of reflected light beams in the wavelength range from 380 nm to 890 nm to sense the light of each of the wavelengths corresponding to UV (380-450 nm), blue (420-500 nm), green (480-580 nm), orange (530-730 nm), red (630-680 nm), far-red (680-760 nm), infrared (780-890 nm) and white (430-760 nm).

The respective peak wavelengths of the wavelength ranges may be UV (about 405 nm), blue (about 457 nm), green (about 527 nm), orange (about 600 nm), red (about 660 nm), far-red (730 nm), infrared (about 860 nm), and white (about 600 nm).

To obtain the light reflection intensity, i.e., light intensity image, in the wavelength range from 380 nm to 890 nm, the light reflection intensity may first be obtained from the magnetic beads at each wavelength ranging from 380 nm to 890 nm.

The detector according to the disclosure may convert the optical signal from the suspension irradiated by the illuminator into an electrical signal and generate an image based on electrical signals.

The left-hand image of FIG. 2 is an image generated by merging the respective images of the reflected light beams at the different wavelengths and reflecting all corresponding light data for each wavelength per pixel.

For example, eight images respectively for the reflected light beams at the different wavelengths corresponding to UV, blue, green, orange, red, far-red, infrared, and white may be captured, and data corresponding to eight wavelengths per pixel may be stored.

The detector according to the disclosure may perform image processing on the received reflected light beams at the different wavelengths.

Image processing may include various types of processing, including noise cancellation, brightness adjustment, sharpness adjustment, or other processing for enhancing image quality, image resizing, and data reformatting.

For example, the per-wavelength images of the disclosure may be merged, generated, and displayed into a single image, as shown in FIG. 2, by obtaining gray images through a monochrome camera and image-processing the obtained per-wavelength images.

To that end, the image shown in FIG. 2 is one generated as a color image by allocating colors to the per-wavelength gray images by way of a known pseudo color representation method, and may display a tiny change in concentration level by reflecting all the light data for each reflected light for the eight wavelengths per pixel.

The left-hand image of FIG. 2 shows a tiny change in concentration level based on the degree of color.

The magnetic beads according to the disclosure may include iron oxide (FeO, Fe₂O₃, Fe₃O₄) and may be red-brown iron oxide particles. It may be seen that the colors of the per-column wells of the plate shown in FIG. 2 are different depending on the content of the red-brown magnetic beads. As the red-brown chromaticity increases, the concentration of the magnetic bead-suspended suspension may increase while as the red-brown chromaticity decreases, the concentration of the magnetic bead-suspended suspension may decrease.

To determine the concentration of the suspension through the chromaticity, in step 114, the light data of the reflected light beams at the different wavelengths is analyzed and, based on the analyzed light data, the concentration of the magnetic beads in the plurality of wells is determined in step 116. In this case, determining the concentration may be determining relative amounts of the magnetic beads per well, as well as determining the precise concentration of the magnetic beads per well.

In the disclosure, to determine the concentration of the magnetic beads per well, a plurality of light beams having different wavelengths are irradiated to the plurality of wells, the plurality of per-wavelength light beams are sensed from the plurality of wells, and data of the sensed reflected light beams at the different wavelengths is analyzed.

To that end, a result of measuring the intensity by analyzing the light data of the reflected light beams at the different wavelengths per well may be shown as in the following table.

### EMBODIMENTS

The inventors identified whether the concentration of the magnetic beads in a magnetic beads-suspended suspension might be determined according to a method of the disclosure.

### Embodiment 1: Preparation for a Suspension in which Magnetic Beads are Suspended

A 96-well plate was filled with a liquid, and magnetic beads were put in. The 96-wells containing the magnetic beads all are filled with the same amount of liquid.

The magnetic beads were put, in grams of 50% of the liquid, in a first column of wells, in grams of 40% of the liquid, in a second column of wells, in grams of 30% of the liquid, in a third column of wells, in grams of 20% of the liquid, in a fourth column of wells, and in grams of 10% of the liquid, in a fifth column of wells.

### Embodiment 2: Selection of Suspension with Normal Concentration Range

Concentration measurement was conducted on each magnetic beads-suspended well. The concentration of the suspension desired in the disclosure is one for the wells in the third column (the wells in which the magnetic beads were put in grams of 30% of the liquid). Accordingly, in the embodiment according to the disclosure, the concentration measured on the third column was set to be in a normal range, and the rest set to be in an error range.

Each well in the third column was irradiated with UV light, blue light, green light, orange light, red light, far-red light, infrared light, and white light, using an illuminator.

As a result, the intensity of the reflected light for each wavelength could be obtained. The obtained intensity of reflected light for each wavelength was set as a reference intensity for each wavelength in the suspension having a concentration in the normal range. Thus, a dynamic range was set for the concentration and the reference intensity for each wavelength.

### Embodiment 3: Determination of Concentration of Magnetic Beads in Suspension

For the accuracy of the experiment, in the 96-well plate, magnetic beads were put in some wells to allow the suspension to have a lower concentration than the concentration set to be in the normal range, in other wells to allow the suspension to have a higher concentration, and in yet other wells to allow the suspension to have the normal concentration.

The 96-well plate was sequentially irradiated with eight wavelengths of light using an illuminator.

Well NO. Table 1 shows per-wavelength intensities of the wells determined to have a normal concentration in the magnetic bead concentration determination according to the disclosure and an average concentration according thereto.

TABLE 1
Well NO 1.

| | | | | | | average |
|---|---|---|---|---|---|---|
| wave- | | wave- | | wave- | | concen- |
| length | intensity | length | intensity | length | intensity | tration |
| UV | 62.414 | AM | 135.511 | IR | 241.876 | 136 |
| B | 50.666 | R | 187.770 | W | 103.751 | mg/L |
| G | 61.827 | FR | 252.176 | | | |

Well NO. Table 2 shows per-wavelength intensities of the wells determined to have an abnormal concentration in the magnetic bead concentration determination according to the disclosure and an average concentration according thereto.

TABLE 2
Well NO 2.

| | | | | | | average |
|---|---|---|---|---|---|---|
| wave- | | wave- | | wave- | | concen- |
| length | intensity | length | intensity | length | intensity | tration |
| UV | 32.212 | AM | 76.211 | IR | 120.456 | 76 |
| B | 25.333 | R | 97.330 | W | 50.241 | mg/L |
| G | 30.423 | FR | 152.98 | | | |

It is apparent to one of ordinary skill in the art that the per-wavelength values and the average concentration in the tables are examples for convenience of description and the disclosure is not limited thereto.

According to an embodiment, the table may be displayed together with the well image of FIG. 2, showing the number (e.g., well NO 1 or NO 2) corresponding to the well selected by the user, per-wavelength intensities, and the average concentration according thereto.

According to an embodiment, the table is of the type to show the per-wavelength intensities for a specific well and the average concentration according thereto, and if the tables are analysis information or result information about each of the plurality of wells, the analysis information or result information for each of the plurality of wells formed in the plate displayed in the table may be stored and managed in any type of database to which the disclosure is applied.

In the table, the average concentration may be calculated to differ based on various factors according to an embodiment, which is described below in detail. Accordingly, there may be various methods for calculating the average concentration in the table according to an embodiment.

In step 114, analysis of the light data of the reflected light beams at the different wavelengths is for measuring the intensities of the reflected light beams at the different wavelengths and determining the concentration of the magnetic beads based on the measured per-wavelength intensities. In other words, determining the magnetic bead concentration in the suspension according to the disclosure is to measure the per-wavelength intensities and determine the magnetic bead concentration based on the measured intensities.

According to an embodiment, the measurement of the magnetic bead concentration may be performed using a light intensity sensor commonly used for measuring the intensity of reflected light. Alternatively, the intensities of the reflected light beams at the different wavelengths may be measured by irradiating a plurality of light beams, measuring the characteristics of the light reflected for the entire spectrum, and comparing the spectrum of the reflected light with the spectrum of the incident light. According to another embodiment, the intensity of the reflected light may be measured as the amplitude which is the intensity per wavelength or as the number of photons, but the disclosure is not limited thereto.

The determination of the multi-layered circuit board may display the normal concentration and the abnormal concentration to be intuitively distinguished as shown in the tables (well NO 1 and well NO 2).

If the result of measuring the light data, i.e., intensity, of the reflected light beams at the different wavelengths from the plurality of wells irradiated with the plurality of per-wavelength light beams for detecting the concentration of the magnetic beads is determined to have a normal concentration, the intensity for each wavelength shown in a specific color, e.g., blue and, when determined to have an abnormal concentration, the intensity is shown in a different color, e.g., red, than the color when it is determined to have the normal color. In this case, blue and red are indicators for distinguishing the states of the concentration, but the disclosure is not limited thereto.

As such, in the magnetic bead concentration determination method according to the disclosure, it may be determined whether the magnetic bead concentration is normal or abnormal by (i) calculating a certain concentration value or concentration range of the magnetic beads in the well or (ii) identifying whether the concentration of the magnetic beads in the well falls within a predetermined concentration range, using the light data of the reflected light beams at the different wavelengths.

In (i) above, the certain concentration value of the magnetic beads of the well may be shown as concentrations in various units according to an embodiment to calculate the concentration of the suspension per well.

For example, the concentration may be shown in various concentration units known to one of ordinary skill in the art, such as the concentration value representing the number of grams (g) of magnetic beads dissolved in 100 g of suspension in %, molar concentration (molarity) using M (=mol/L) through the amount of substance of magnetic beads dissolved in a unit volume of suspension, or volume percent which is the number of ml of magnetic beads dissolved in 100 ml of suspension, and the concentration values in such units are all calculated based on the intensity of the reflected light per wavelength.

The concentration value or concentration range calculated according to the disclosure may be shown in the unit selected from among the above-described units, but it is apparent to one of ordinary skill in the art that the disclosure is not limited thereto.

The concentration values or concentration ranges of the suspension respectively for the plurality of wells may be displayed as individual values, and it may be determined whether the magnetic bead concentration is normal based on (i) above.

Specifically, it may be identified whether the concentration values or concentration ranges based on the respective intensities of the reflected light beams at the different wavelengths fall within intensities or intensity ranges respectively previously provided for the wavelengths, and it may be determined that the magnetic beads in the wells fall within a desired concentration range and that the concentration is normal (a) if all of the reflected light beams at the different wavelengths fall within the intensity ranges, (b) if a predetermined number of, or more, reflected light beams at the different wavelengths fall within the intensity ranges, or (c) if reflected light beams having two or more pre-selected wavelengths fall within the intensity ranges.

For example, if the concentration values for the plurality of light beams having different wavelengths are calculated as follows (units are omitted for convenience of description),
UV: 62, blue: 50, green: 61, orange: 135, red: 187, far-red: 252, infrared: 241, white: 103

According to an embodiment, to determine the concentration of magnetic beads using (a), the magnetic bead concentration is compared with a reference concentration value or reference concentration range previously provided for each wavelength and, if the reference is reached for each wavelength, it may be determined that the magnetic bead concentration falls within a desired concentration range.

According to another embodiment, to determine the concentration of magnetic beads using (b), the magnetic bead concentration is compared with a reference concentration value or reference concentration range previously provided for each wavelength and, if a preset number of, or more, wavelengths of reflected light beams reach the reference, it may be determined that the magnetic bead concentration falls within a desired concentration range. Here, the preset number or more may include at least one or 2, 3, 4, 5, 6, or 7.

According to another embodiment, to determine the concentration of magnetic beads using (c), the magnetic bead concentration is compared with a reference concentration value or reference concentration range previously provided for each wavelength and, if a specific wavelength of reflected light reaches the reference, it may be determined that the magnetic bead concentration falls within a desired concentration range. In this case, the specific wavelength may be a wavelength in a wavelength range in which the color of the magnetic beads may be expressed more clearly, and may be variably selected depending on the color of the object irradiated with light.

Alternatively, to determine the concentration of the magnetic beads based on (i) above, the average of the concentration values or concentration ranges of the reflected light beams of all the wavelengths may be calculated using (a) and, based thereupon, the magnetic beads in the well may be determined to fall within the desired concentration range.

The average of the concentration values or concentration ranges of a preset number of, or more, reflected light beams may be calculated using (b) and, based thereupon, the magnetic beads in the well may be determined to fall within the desired concentration range.

The average of the concentration values or concentration ranges of the reflected light beam having a specific wavelength may be calculated using (c) and, based thereupon, the magnetic beads in the well may be determined to fall within the desired concentration range.

Alternatively, weights may be set for the specific wavelengths of reflected light in each embodiment, and their average may be obtained, and based thereupon, the magnetic beads of the well may be determined to fall within the desired concentration range.

Accordingly, the embodiments set forth herein are provided for illustrative purposes, but not to limit the scope of the disclosure, and should be appreciated that the scope of the disclosure is not limited by the embodiments.

Then, in the magnetic bead concentration determination method according to the disclosure, to determine whether the magnetic bead concentration is normal or abnormal, (ii) above may determine the concentration of the magnetic beads by identifying whether the concentration of the magnetic beads in the well falls within a predetermined concentration range, using the light data of the reflected light beams at the different wavelengths.

Specifically, certain concentration values or certain concentration ranges for the respective intensities of the reflected light beams at the different wavelengths may be standardized and previously provided, it may be identified whether the concentration values or concentration ranges based on the sensed intensities fall within the intensities or intensity ranges having the previously provided standardized concentration values or concentration ranges, and it may be determined that the magnetic beads in the wells fall within a desired concentration range and that the concentration is normal (a) if all of the reflected light beams at the different wavelengths fall within the intensity ranges, (b) if a predetermined number of, or more, reflected light beams at the different wavelengths fall within the intensity ranges, or (c) if reflected light beams having two or more pre-selected wavelengths fall within the intensity ranges.

For example, if the intensities for the sensed plurality of light beams having different wavelengths are measured as follows,
UV: 62, blue: 50, green: 61, orange: 135, red: 187, far-red: 252, infrared: 241, white: 103

Whether the light data of the sensed reflected light beams at the different wavelengths falls within the previously provided intensity value or intensity range is determined based on (a), (b), or (c) above. In this case, the intensity range may correspond to a range of the intensity value per wavelength, which has the previously provided standardized concentration value or concentration range, plus ±10, 20, 30, or more or less.

The identification of whether the concentration of the magnetic beads in the well falls within a predetermined concentration range in (ii) above may be performed using a standard curve of per-wavelength reflected light beam intensities for magnetic beads for a plurality of known concentrations.

Per-wavelength intensities are measured for suspensions having different concentrations in which the magnetic bead concentration is known, and a standard curve graph is created. For example, the graph is created, with the concentrations of the suspensions set to 0, 10, 20, 30, 40, 50, and 60 mg/L on the x-axis, and the intensity of reflected light for each wavelength for each concentration set on the y-axis.

In the disclosure, a reference value range to be linearized using data is determined by linear regression using the standard curve.

If the respective intensities of the reflected light beams at the different wavelengths using the determined reference value range are included by performing (i), (ii), or (iii), the magnetic beads in the well are determined to fall within the desired concentration range.

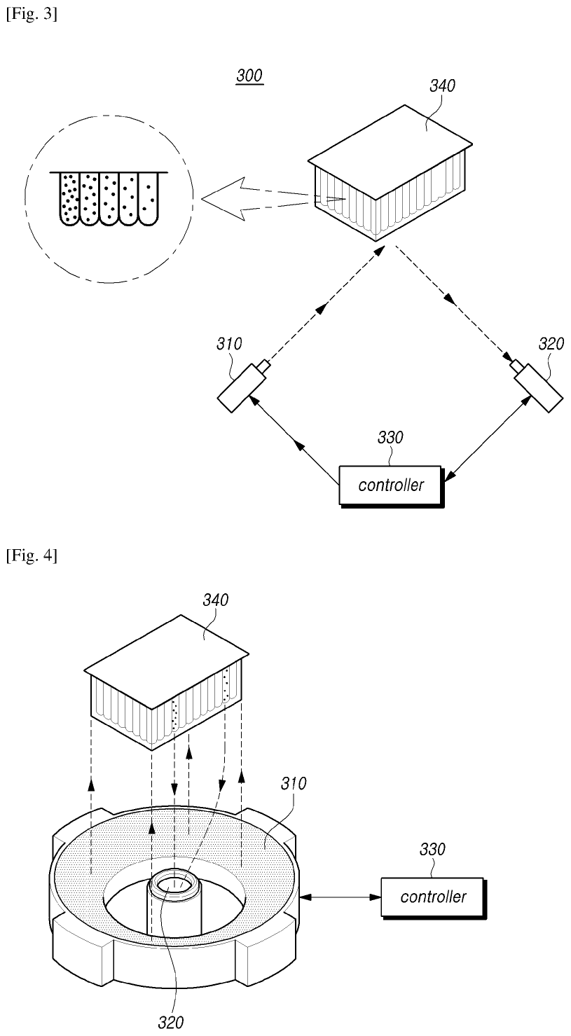

FIGS. 3 and 4 are views illustrating a configuration of a system 300 for implementing a method for determining a magnetic bead concentration in a suspension according to the disclosure.

Although the system 300 according to the disclosure, shown in FIGS. 3 and 4, is configured so that the plate 340 is irradiated and sensed from thereunder, the disclosure is not limited thereto, and various changes may be made to the positions of the illuminator 310 and the detector 320 as long as the entire plate 340 is able to be irradiated, and the light reflected from the wells in the plate can be sensed.

Further, although the illuminator 310 and the detector 320 of the system according to the disclosure are positioned preferably as shown in FIG. 4, the illuminator 310 and the detector 320 according to the disclosure are not limited thereto, and various changes may be made thereto to be suited for the purpose of the disclosure, as the illuminator 310 and the detector 320 distributed and positioned as shown in FIG. 3.

Referring to FIG. 3, a system 300 according to the disclosure includes an illuminator 310, a detector 320, and a controller 330.

The illuminator 310 irradiates a plurality of light beams having different wavelengths to the suspension.

Although the suspension according to the disclosure is a magnetic bead-suspended suspension, the disclosure is not limited thereto, but various changes may be made thereto to be suited for the purpose of inspecting the content of particles, e.g., magnetic beads, dispersed in a solution.

The plurality of light beams are selected from among light beams reflectable by the magnetic beads.

According to an embodiment, the illuminator 310 according to the disclosure may irradiate different wavelengths of light to the plurality of wells.

According to an embodiment of the disclosure, the illuminator 310 may irradiate a single wavelength of white light to the plurality of wells.

The detector 320 senses reflected light beams at the different wavelengths from the suspension to which the plurality of light beams are irradiated by the illuminator.

The detector 320 according to the disclosure may be a colorimeter, and the colorimeter may include a monochrome camera and a red-green-blue (RGB) color camera.

For example, when the detector is an RGB color camera, the illuminator may irradiate a single wavelength of white light and may sense the irradiated white light through filters of red light, green light, and blue light.

When the detector is a monochrome camera, the illuminator may sense each of light beams including UV light, blue light, green light, orange light, red light, far-red light, infrared light, and white light.

The disclosure is to sense, through the detector 320, reflected light beams at the different wavelengths from the plurality of wells in response to the light irradiated from the illuminator 310 to the plurality of wells, measure the intensities of the sensed reflected light beams at the different wavelengths, and determine the concentration of the magnetic beads per well based on the measured intensities.

To that end, the controller 330 according to the disclosure controls the illuminator 310 to irradiate the different wavelengths of light according to on-off combinations, drives the detector 320 according to a control signal synchronized with the control signal, analyzes light data of the reflected light beams at the different wavelengths sensed by the detector 320 to measure the intensities of the reflected light beams at the different wavelengths, and determines the concentration of the suspension.

According to an embodiment, if per-wavelength filters are used, the illuminator 310 may irradiate the different wavelengths of light to be distinguished from each other, through one light irradiation without the on-off combinations for the per-wavelength light. In such a case, the detector 320 may detect the once sensed reflected light per wavelength through the filters.

According to an embodiment, to determine whether the magnetic bead concentration is normal or abnormal, the controller 330 (i) calculates a certain concentration value or concentration range of the magnetic beads of the wells or (ii) identifies whether the concentration of the magnetic beads of the wells falls within a predetermined concentration range using the light data of the reflected light beams at the different wavelengths.

To determine whether the magnetic bead concentration is normal based on (i) above, the controller 330 may identify whether the concentration values or concentration ranges based on the respective intensities of the reflected light beams at the different wavelengths fall within intensities or intensity ranges respectively previously provided for the wavelengths, and may determine that the magnetic beads in the wells fall within a desired concentration range and that the concentration is normal (a) if all of the reflected light beams at the different wavelengths fall within the intensity ranges, (b) if a predetermined number of, or more, reflected light beams at the different wavelengths fall within the intensity ranges, or (c) if reflected light beams having two or more pre-selected wavelengths fall within the intensity ranges.

To determine the concentration of the magnetic beads by identifying whether the concentration of the magnetic beads in the well falls within the predetermined concentration range in (ii) using the light data of the reflected light beams at the different wavelengths so as to determine whether the magnetic bead concentration is normal or abnormal, the controller 330 may standardize and previously provide certain concentration values or certain concentration ranges for the respective intensities of the reflected light beams at the different wavelengths, identify whether the concentration values or concentration ranges based on the sensed intensities fall within the intensities or intensity ranges having the previously provided standardized concentration values or concentration ranges, and determine that the magnetic beads in the wells fall within a desired concentration range and that the concentration is normal (a) if all of the reflected light beams at the different wavelengths fall within the intensity ranges, (b) if a predetermined number of, or more, reflected light beams at the different wavelengths fall within the intensity ranges, or (c) if reflected light beams having two or more pre-selected wavelengths fall within the intensity ranges.

According to an embodiment, the controller 330 may identify whether the concentration of the magnetic beads in the well falls within the predetermined concentration range using a standard curve of the intensities of the reflected light beams at the different wavelengths for the magnetic beads for a plurality of known concentrations.

According to an embodiment, the system 300 according to the disclosure may include an automatic vision inspection system.

The automatic vision inspection system is provided with a plate formed of a plurality of wells, a conveyor on which the plate is positioned and conveyed, an illuminator, and a detector and includes an image acquisition device for vision inspection for obtaining an image for vision inspection and a computer for vision inspection that analyzes, inspects, and judges the image data received from the image acquisition device for vision inspection, stores and manages (database) the image information for vision inspection, and stores and manages the vision inspection result information. The computer for vision inspection may have gray data values and determine the image obtained from the image acquisition device for vision inspection by a reference value set for inspection and determination or may have data values for RGB components and determine the obtained image by the reference value set for inspection and determination.

When an element “comprises,” “includes,” or “has” another element, the element may further include, but rather than excluding, the other element, and the terms “comprise,” “include,” and “have” should be appreciated as not excluding the possibility of presence or adding one or more features, numbers, steps, operations, elements, parts, or combinations thereof. All the scientific and technical terms as used herein may be the same in meaning as those commonly appreciated by a skilled artisan in the art unless defined otherwise. It will be further understood that terms, such as those defined dictionaries, should be interpreted as having a meaning that is consistent with their meaning in the context of the relevant art and will not be interpreted in an idealized or overly formal sense unless expressly so defined herein.

The above-described embodiments are merely examples, and it will be appreciated by one of ordinary skill in the art various changes may be made thereto without departing from the scope of the disclosure. Accordingly, the embodiments set forth herein are provided for illustrative purposes, but not to limit the scope of the disclosure, and should be appreciated that the scope of the disclosure is not limited by the embodiments. The scope of the disclosure should be construed by the following claims, and all technical spirits within equivalents thereof should be interpreted to belong to the scope of the disclosure.

While embodiments of the disclosure have been described above, it will be apparent to one of ordinary skill in the art that the specific techniques are merely preferred embodiments and the scope of the disclosure is not limited thereto. Thus, the scope of the disclosure is defined by the appended claims and equivalents thereof.

### CROSS-REFERENCE TO RELATED APPLICATION

This application claims priority from Korean Patent Application No. 10-2021-0059334, filed on May 7, 2021, in the Korean Intellectual Property Office, the disclosure of which is incorporated herein by reference in its entirety.

1. A method for determining a magnetic bead concentration in a suspension, the method comprising:
irradiating a plurality of light beams having different wavelengths to a plurality of wells containing a suspension in which magnetic beads are suspended;
sensing reflected light beams at the different wavelengths from the plurality of wells using a detector including a light sensor; and
determining concentrations of the magnetic beads in the plurality of wells by analyzing light data of the sensed per-wavelength reflected light beams.

2. The method of claim 1, wherein sensing the reflected light beams at the different wavelengths includes measuring intensities of the reflected light beams at the different wavelengths.

3. The method of claim 1, wherein the plurality of light beams are selected from among light beams reflectable by the magnetic beads.

4. The method of claim 3, wherein the plurality of light beams are two or [claim 4] more light beams selected from among ultraviolet (UV) light, blue light, green light, orange light, red light, far-red light, infrared light, and white light, and wherein the plurality of light beams are sequentially irradiated.

5. The method of claim 1, wherein determining the concentrations of the magnetic beads includes (i) calculating a certain concentration value or a concentration range of the magnetic beads in the wells or (ii) identifying whether the concentrations of the magnetic beads in the wells fall within a predetermined range, using the light data of the reflected light beams at the different wavelengths.

6. The method of claim 1, wherein analyzing the light data of the sensed reflected light beams at the different wavelengths includes determining the concentration of the magnetic beads in the plurality of wells, using a standard curve of per-wavelength reflected light intensities for magnetic beads for a plurality of known concentrations.

7. The method of claim 2, further comprising identifying whether respective intensities of the reflected light beams at the different wavelengths fall within reference intensity ranges respectively provided for the wavelengths and determining that the magnetic beads in the wells fall within a desired concentration range, (i) if all of the reflected light beams at the different wavelengths fall within the reference intensity ranges, (ii) if a predetermined number of, or more, reflected light beams at the different wavelengths fall within the reference intensity ranges, or (iii) if reflected light beams having two or more pre-selected wavelengths fall within the reference intensity ranges.

8. The method of claim 1, wherein the detector is a colorimeter.

9. The method of claim 1, wherein the detector senses the reflected light beams per wavelength by a monochrome camera and provides an image for the sensed reflected light beam per wavelength.

10. The method of claim 1, wherein irradiating the plurality of light beams includes irradiating the light beams having the different wavelengths according to on-off combinations by a controller, and
wherein sensing the reflected light beams at the different wavelengths includes driving the detector in synchronization with the irradiation by the controller.

11. The method of claim 1, wherein the magnetic beads include iron oxide.

12. The method of claim 1, wherein the magnetic beads are red-brown iron oxide particles.

13. The method of claim 1, wherein the magnetic beads have a size of 0.1 μm to 6 μm.

14. A system for determining a concentration in a suspension, the system comprising:
an illuminator irradiating a plurality of light beams having different wavelengths to the suspension;
a detector sensing reflected light beams at the different wavelengths from the suspension to which the plurality of light beams are irradiated by the illuminator; and
a controller controlling the illuminator to irradiate the light beams having the different wavelengths according to on-off combinations, driving the detector, measuring intensities of the reflected light beams at the different wavelengths by analyzing light data of the reflected light beams at the different wavelengths sensed by the detector, and determining a concentration of the suspension.

15. The system of claim 14, wherein the controller calculates a certain concentration value or a concentration range of the magnetic beads in the wells or identifies whether the concentrations of the magnetic beads in the wells fall within a predetermined range, using the light data of the reflected light beams at the different wavelengths.

16. The system of claim 15, wherein the controller uses a standard curve of per-wavelength reflected light intensities for magnetic beads for a plurality of known concentrations.

17. The system of claim 15, wherein the controller identifies whether respective intensities of the reflected light beams at the different wavelengths fall within reference intensity ranges respectively provided for the wavelengths and determines that the magnetic beads in the wells fall within a desired concentration range (i) if all of the reflected light beams at the different wavelengths fall within the reference intensity ranges, (ii) if a predetermined number of, or more, reflected light beams at the different wavelengths fall within the reference intensity ranges, or (iii) if reflected light beams having two or more pre-selected wavelengths fall within the reference intensity ranges.

18. The system of claim 14, wherein the suspension is a solution in which magnetic beads are suspended.
